(19) United States
(12) Patent Application Publication
(10) Pub. No.: US 2008/0287719 A1
(43) Pub. Date: Nov. 20, 2008

(54) Process for para-xylene production from light aliphatics

(76) Inventors: Deng-Yang Jan, Elk Grove Village, IL (US); Stanley J. Frey, Palatine, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/027,149

(22) Filed: Dec. 30, 2004

(51) Int. Cl. C07C 2/52

(52) U.S. Cl. 585/418

(57) ABSTRACT

The subject process obtains a high yield of high-purity para-xylene from a butene dimer feed. The process may include dimerization of isobutene to obtain a butene dimer comprising C₈ iso-olefins and isoparaffins, aromatization of the dimerized C₈ product, and recovery of high-purity para-xylene from the dimerized product by low-intensity crystallization. Aromatization is effected using a non-acidic, non-zeolitic catalyst. Each of the processing steps may be tailored to the overall objective of high para-xylene yield from a relatively inexpensive feedstock.

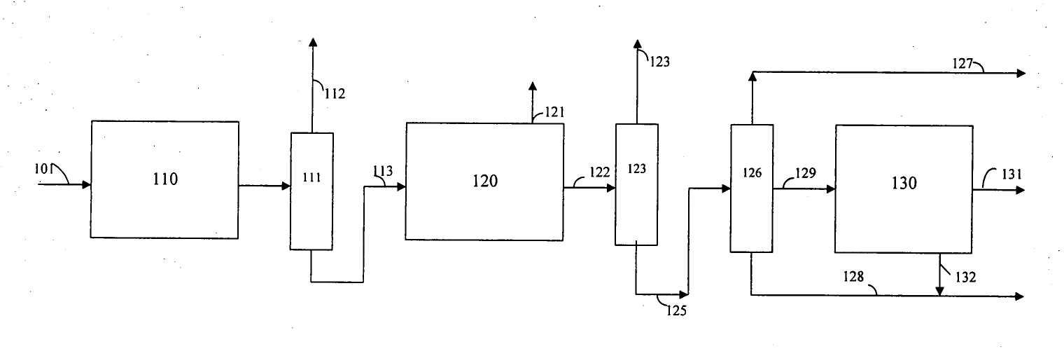

### FIELD OF THE INVENTION

The present invention relates to the field of aromatic petrochemicals. More specifically, the invention relates to the production of para-xylene from light aliphatic hydrocarbons.

### BACKGROUND OF THE INVENTION

Para-xylene is an important intermediate in the chemical and fiber industries. Terephthalic acid derived from para-xylene is used to produce polyester fabrics and other articles which are in wide use today.

Usually para-xylene is produced, in a series of steps, from naphtha fractionated from crude oil. Naphtha is hydrotreated and reformed to yield aromatics, which then are fractionated to separate typically benzene, toluene and C₈ aromatics comprising xylenes from C₉ and heavier aromatics. Toluene and C₉ aromatics may be disproportionated to yield additional xylenes. Xylene isomers, with the usual priority being para-xylene, are separated from the mixed C₈-aromatics stream using one or a combination of adsorptive separation, crystallization and fractional distillation, with adsorptive separation being most widely used in newer installations for para-xylene production. Other C₈ isomers may be isomerized and returned to the separation unit to yield additional para-xylene.

Although low-value light aliphatics such as butanes and butenes offer a substantial theoretical margin for the production of para-xylene, practical processes to effect this conversion have not been available to date. Butane dehydrogenation and dimerization plus aromatization to yield primarily octane isomers is taught in U.S. Pat. Nos. 5,847,252, 5,856,604 and 6,025,533. U.S. Pat. No. 4,367,356 discloses a combination of butene dimerization and alkylation to obtain C₈ hydrocarbons. These patents, whose relevant teachings are incorporated herein by reference, do not disclose the production of para-xylene.

In the Journal of Catalysis 1 (1962), pp. 313-328, Pines and Csicsery disclose the aromatization of trimethylpentanes to xylenes, using a nonacidic chromia-alumina catalyst; 2,2,4-trimethylpentane formed only para-xylene. In the proceedings of the 1962 Radioisotopes Physical Science Industrial Process Conference at pages 205-216, Cannings et al. teach dehydrocyclization of 2,2,4-trimethylpentane over a potassium- and cerium-promoted chromia-alumina catalyst to selectively yield para-xylene. British Patent 795,235 teaches the manufacture of para-xylene from 2,4,4-trimethylpentene using a catalyst comprising a Group VI-A oxide, exemplified as a series of chromia-containing catalysts. U.S. Pat. No. 3,202,725 discloses dehydrogenation of isobutane and recycle di-isobutylene using a chromia-alumina catalyst to yield para-xylene and isobutene, plus dimerization of the isobutene using a silica-alumina, phosphoric acid or sulfuric acid catalyst to yield primarily di-isobutylene recycle. U.S. Pat. No. 3,462,505 discloses the dehydrocyclization of 2,2,4-trimethylpentane to yield para-xylene using a catalyst comprising chromia, magnesia and an alkali metal on activated alumina. U.S. Pat. No. 3,766,291 discloses disproportionation of amylene to 2,5-dimethylhexene, which then is selectively converted to para-xylene over a catalyst comprising a Group II metal (exemplified by Zn) aluminate, tin-group metal, and Group VIII metal. U.S. Pat. No. 4,910,357 teaches the aromatization of dimethylhexanes, especially those contained in alkylate, using a catalyst comprising a dehydrogenation metal and a nonacidic crystalline support containing Sn, Tl, In and/or Pb. U.S. Pat. No. 6,177,601 B1 teaches aromatization of 2,5-dimethylhexane to selectively produce para-xylene, using a nonacidic L-zeolite catalyst. U.S. Publication 2004/0044261A1 teaches production of para-xylene from a feedstock rich in C₈ isoalkanes or isoalkenes using a catalyst comprising a molecular sieve, Group VIII metal and two or more of Si, Al, P, Ge, Ga and Ti. U.S. Publication 2004/0015026 discloses the manufacture of para-xylene from 2,2,4-trimethylpentane using a catalyst comprising chromium. It should be noted that chromium, as a catalyst constituent, is a toxic element.

None of the above references disclose the selective process of the present invention. To date, therefore, the art does not disclose a practical process for the production of para-xylene from light hydrocarbons.

### BRIEF DESCRIPTION OF THE INVENTION

In a broad embodiment this invention is a process for the production of para-xylene from an aliphatic butene dimer. The process converts a feed stream comprising one or both of C₈ olefins and paraffins in an aromatization zone in which olefins and paraffins are converted by contact with a non-zeolitic, nonacidic aromatization catalyst at aromatization conditions to yield xylenes having a high concentration of para-xylene.

In a more specific embodiment this invention is a process combination for the production and recovery of para-xylene from an aliphatic butene dimer. The process converts a feed stream comprising one or both of C₈ olefins and paraffins in an aromatization zone in which olefins and paraffins are converted by contact with a non-zeolitic, nonacidic aromatization catalyst at aromatization conditions. Effluent from the aromatization zone is fractionated to recover a mixed C₈-aromatics stream having a high concentration of para-xylene, which passes to a separation zone for separation of para-xylene from residual C₈ aromatics. Preferably the separation zone comprises a single-stage crystallizer.

In a more specific embodiment, an isobutene-containing stream is processed in a dimerization zone. In the dimerization zone, the isobutene-containing stream contacts a dimerization catalyst at dimerization conditions to recover a butene dimer comprising branched C₈ aliphatics. The aliphatic-containing butene dimer, comprising one or both of C₈ olefins and paraffins, passes to an aromatization zone in which olefins and paraffins are converted by contact with a non-zeolitic, nonacidic aromatization catalyst at aromatization conditions. Effluent from the aromatization zone is fractionated to recover mixed C₈-aromatics stream which passes to a separation zone for separation of para-xylene from residual C₈ aromatics. Preferably the separation zone comprises a single-stage crystallizer.

An integrated process combination comprising the invention effectively converts light aliphatic hydrocarbons to obtain a high yield of valuable para-xylene.

### BRIEF DESCRIPTION OF THE DRAWINGS

The FIGURE is a schematic process flow diagram of the invention when processing an isobutene concentrate to yield para-xylene.

### DETAILED DESCRIPTION OF THE INVENTION

A process combination and individual operational steps are described in conjunction with the FIGURE. The FIGURE shows only those portions of the process that are necessary to gain an understanding of the invention and the means of integrating the different process steps that comprise the invention. Further details related to heaters, coolers, exchangers, valves, control means, pumps, compressors, and other necessary processing equipment are well known to those skilled in the art and not described in detail unless necessary for an understanding of the invention. Also, this description does not exclude from the inventive concept other embodiments which may result from the modification of the descriptions by a skilled routineer.

The FIGURE illustrates an embodiment of the process combination of the invention, including a dimerization zone to produce an aliphatic butene dimer, when processing a feed rich in isobutene. In the dimerization zone 110, the isobutene-rich feed 101 passes through a series of dimerization reactors. Zone 110 is divided into multiple reactor stages in order that the dimerization reaction temperature can be controlled by injection of quench between stages. The isobutene is selectively dimerized to form primarily branched C₈ olefins and/or paraffins. Effluent from the dimerization reactors is stabilized in unit 111 to separate C₄ and lighter products in stream 112, with stabilized butene dimer passing to the aromatization zone 120 via line 113.

The aromatization zone converts the butene dimer from zone 110 to yield a high proportion of para-xylene. The aromatization zone 120 typically uses a plurality of reactors arranged in series with the entire feed passing through each reactor. Since the aromatization reaction is highly endothermic, a series of reactors with reheating between each reactor permits greater control of the processing temperature. Hydrogen generated by aromatization is circulated within the aromatization zone, with a net hydrogen stream recovered via line 121. Liquid effluent from aromatization passes via line 122 to unit 123 to separate C₄ and lighter products in line 124. Debutanized product from 123 passes in line 125 to unit 126 for further separation of products. Unit 126 may be a sidestream fractionator as shown, or it may comprise two or more fractionating columns. In any event, a stream comprising C₅ to C₇ hydrocarbons is removed from the process in line 127 and a stream comprising C₉ and heavier hydrocarbons is removed in line 128 in order to provide a para-xylene concentrate 129 as feed to para-xylene-recovery zone 130.

Separation zone 130 may comprise any suitable process to recover para-xylene of the desired purity, usually >99.7% purity, from the concentrate in line 129, including without limitation one or more of continuous adsorption, pressure-swing adsorption, fractionation and crystallization. Single-stage crystallization is preferred as a relatively inexpensive technique to separate high-purity para-xylene in line 131 from a feed rich in the para-isomer. The reject stream in line 132 is rich in other C₈-aromatics isomers. Streams 127, 128 and 132 all are suitable components for gasoline blending, being rich in high-octane aromatics.

Suitable dimerization zones to prepare feed for an aromatization zone of this invention may be known by a variety of names and employ one or more of several catalyst types. Other names for the dimerization zone include oligomerization, catalytic condensation and catalytic polymerization. The use of resin catalysts for effecting dimer production is described, for example, in U.S. Pat. Nos. 4,100,220; 4,215,011; and 4,302,356. The use of a layered molecular sieve for isobutene oligomerization is taught in U.S. Pat. No. 6,649,802 B1. U.S. Pat. No. 6,689,927 B1 discloses oligomerization of isobutene using a solid phosphoric acid catalyst. The applicable teachings of all of the above references in this paragraph are incorporated herein by reference thereto. An effective dimerization zone provides a high yield of iso-octenes and iso-octanes having a high concentration of one or more of 2,4,4-trimethylpentene, 2,2,4-trimethylpentane, 2,5-dimethylhexene and 2,5-dimethylhexane in the product from the zone.

The dimerization zone alternatively may comprise alkylation of isobutane with butenes to provide a suitable feedstock for the aromatization step; the integration of an alkylation unit with a dehydrogenation unit is described, for example, in U.S. Pat. No. 4,275,255, incorporated herein by reference thereto. An isobutane-containing stream and the dehydrogenation effluent stream or the dehydrogenation product stream containing isobutylene and isobutene are contacted in the dimerization zone with an alkylation catalyst to produce a butene dimer which comprises a high concentration of C₈ isoparaffins. One typical product from the alkylation of isobutene with isobutane had the following yield structure in wt.-%:

| | |
|---|---|
| Lighter than C₈ | 3.6 |
| 2,2,4-trimethylpentane | 67.3 |
| 2,3,4-trimethylpentane | 13.0 |
| 2,3,3-trimethylpentane | 7.2 |
| Dimethylhexanes | 3.5 |
| Heavier than C₈ | 5.4 |

A dimerization catalyst preferably is disposed in fixed beds within the dimerization zone in what is known as a chamber-type reactor structure. In a chamber-type reactor, the reactants flow through one or more fixed catalyst beds. The temperature gradient within the reactor from the exothermic dimerization reaction is controlled by recycling relatively inert hydrocarbons which act as a heat sink. The unreacted isobutane from the dehydrogenation zone supplies a large proportion of the inert hydrocarbons that act as the heat sink. The temperature gradient within the dimerization reaction zone also may be controlled by the use of a quench material between the catalyst beds. As a secondary purpose, the quench material can provide a flushing function to inhibit the development of coke and the deactivation of coke in the deactivation of the catalyst within the reaction zones. Unconverted isobutene, containing unconverted butanes from the dehydrogenation zone, from stabilization of the butene dimer may be used as quench. Higher molecular weight quench material may be used within the dimerization reaction zones to flush the catalyst and preventing coke production. The recycle of such materials as the C₅ to C₇ byproduct from the aromatization zone can also improve selectivity of the dimerization zone to produce the desired C₈ products. Since the higher molecular weight materials have benefits beyond use as a quench, it can be beneficial to add all or a portion of such material to the inlet of dimerization reactor with the feed.

A particularly preferred dimerization catalyst is a cationic resin catalyst such as the Amberlyst series (for example, Amberlyst 15) as produced by Rohm & Haas. The present process preferably is carried out in a substantially vertical fixed catalyst bed; for example, a bed of cation exchange resin supported in a vertical reactor. The flow in the reactor may be upward or downward, with downflow being preferred. Generally, the liquid hydrocarbon and an optional water, ether and/or alcohol cofeed may pass through a single line or separate lines into the reactor. A preferred cofeed concentration is an equivalent of 0.001 to 1 mol of t-butanol per mol of isobutene.

A range of yields may be effected by varying conversion, as illustrated by the following yields from a feedstock containing 43.5 wt.-% isobutene and 1.5 wt.-% normal butene with the balance being primarily butanes:

| | | | |
|---|---|---|---|
| Isobutene conversion, % | 49.6 | 68.1 | 83.3 |
| Hydrocarbon product distribution, wt.-%: | | | |
| C₇− | 0.26 | 0.46 | 0.44 |
| Di-isobutene | 90.0 | 84.4 | 82.5 |
| Other C₈ | 0.49 | 0.82 | 1.03 |
| C₉ and heavier (~80% tri-isobutene) | 9.25 | 14.3 | 16.0 |

The product also contained about 0.5 wt.-% ethers.

Preferred dimerization conditions when utilizing a resin catalyst comprise a liquid hourly space velocity (LHSV) with respect to isobutene of 0.1 to 3.0, with LHSV of 0.5 to 2.0 being preferred, based on fresh feed (i.e., excluding recycle). Reaction temperature generally ranges between 55° and 160° C., with a preferred temperature range of about 100° to 130° C. There may be a temperature gradient through the bed, which preferably is no greater than about 10° to 25° C. The reaction is carried out under sufficient pressure to maintain a liquid phase system, e.g., 1.5 to 2.5 MPa.

A well known alternative catalyst for the dimerization process is a solid phosphoric acid (SPA) catalyst. The SPA catalyst refers to a solid catalyst that contains as a principal ingredient an acid of phosphorus such as ortho-, pyro- or tetraphosphoric acid. The catalyst is normally formed by mixing the acid of phosphorus with a siliceous solid carrier to form a wet paste. This paste may be calcined and then crushed to yield catalyst particles where the paste may be extruded or pelleted prior to calcining to produce more uniform catalyst particles. The carrier is preferably a naturally occurring porous silica-containing material such as kieselguhr, kaolin, infusorial earth, and diatomaceous earth. A minor amount of various additives such as mineral talc, fuller's earth, and iron compounds including iron oxide may be added to the carrier to increase its strength and hardness. The combination of the carrier and the additives preferably comprises about 15-30% of the catalyst, with the remainder being the phosphoric acid. The additive may comprise about 3-20% of the total carrier material. Variations from this such as a lower phosphoric acid content are however possible. Further details as to the composition and production of SPA catalysts may be obtained from U.S. Pat. Nos. 3,050,472; 3,050,473; and 3,132,109 and from other references.

When utilizing the alternative SPA catalyst, dimerization conditions comprise a preferred temperature in the reaction zone of from about 90° to 260° C., and more typically in a range of from about 150° to 230° C. Pressures within the dimerization reaction zone will usually be in a range of from 200 kPa to 8 MPa, and more typically in a range of from 1.4 to 4 MPa. Steam or water may be fed into the reactor to maintain the desired water content in the preferred catalyst.

Effluent from the dimerization zone is stabilized to separate overhead unconverted isobutene along with butanes and lighter hydrocarbons. The stabilizer overhead may be recycled to the dimerization zone for further conversion of the isobutene as well as for temperature control of the reaction. The stabilized butene dimer, comprising one or both of iso-octenes and iso-octanes, comprises the feed to the aromatization zone.

It is within the scope of the present invention that part or all of the butene dimer is processed in a dimer hydrogenation zone before being passed to the aromatization zone. Suitable conditions and catalysts for dimer hydrogenation are taught in U.S. Pat. Nos. 5,847,252; 5,856,604 and 6,025,533, incorporated herein by reference thereto. The hydrogenation zone would yield a hydrogenated dimer comprising 2,2,4-trimethylpentane and 2,5-dimethylhexane along with unconverted butene dimer as feed to the aromatization zone. This optional hydrogenation would also generate part if not all of the heat required to convert partially or fully hydrogenated butene dimer to aromatics. Preferably, however, the stabilized butene dimer is not fully hydrogenated before passing to the aromatization zone.

The butene dimer passing to the aromatization zone comprises a high concentration of one or more of 2,4,4-trimethylpentene, 2,2,4-trimethylpentane, 2,5-dimethylhexene and 2,5-dimethylhexane. The present process is particularly effective for the aromatization of butene dimer that is not fully hydrogenated, namely a feed stream containing trimethylpentenes which are less readily converted in processes of the known art.

The aromatization process may be effected in a reactor section comprising one reactor or in multiple reactors with provisions known in the art to adjust inlet temperatures to individual reactors. The feed may contact the catalyst system in each of the respective reactors in either upflow, downflow, or radial-flow mode. Since the preferred aromatization process operates at relatively low pressure, the low pressure drop in a radial-flow reactor favors the radial-flow mode. As the predominant dehydrocyclization reaction is endothermic, the reactor section generally will comprise two or more reactors with interheating between reactors to compensate for the endothermic heat of reaction and maintain dehydrocyclization conditions.

The reactor section usually is associated with catalyst-regeneration options known to those of ordinary skill in the art, such as: (1) a semiregenerative unit containing fixed-bed reactors maintains operating severity by increasing temperature, eventually shutting the unit down for catalyst regeneration and reactivation; (2) a swing-reactor unit, in which individual fixed-bed reactors are serially isolated by manifolding arrangements as the catalyst become deactivated and the catalyst in the isolated reactor is regenerated and reactivated while the other reactors remain on-stream; (3) a moving-bed reactor with continuous catalyst withdrawal, regeneration, reactivation and substitution of the reactivated catalyst, permitting higher operating severity by maintaining high catalyst activity through regeneration cycles of a few days; (4) a hybrid system with semiregenerative and continuous-regeneration provisions in the same unit; (5) an ebullated-bed reactor with continuous catalyst withdrawal and regeneration; (6) a continuously stirred tank reactor; or (7) a riser-reactor reforming process, generally associated with a fluidized reactor and continuous catalyst regeneration according to U.S. Pat. No. 5,565,090 which is incorporated herein by reference. The preferred embodiment of the present invention is a moving-bed reactor with continuous catalyst regeneration.

An aromatization catalyst preferably incorporates porous, adsorptive, high-surface-area materials as a support. Within the scope of the present invention are refractory supports containing one or more of: (1) refractory inorganic oxides such as alumina, silica, titania, magnesia, zirconia, chromia, thoria, boria or mixtures thereof, (2) synthetically prepared or naturally occurring clays and silicates, which may be acid-treated; (3) spinels such as MgAl₂O₄, FeAl₂O₄, ZnAl₂O₄; and (4) combinations of materials from one or more of these groups. Preferably the aromatization catalyst of the present invention is non-zeolitic, i.e., the catalyst has a substantial absence of zeolitic aluminosilicates or other microporous crystalline material. By “substantial absence” is meant that the concentration of molecular sieves is less than about 1.0 wt.-%.

The preferred support optimally comprises a porous, adsorptive, high-surface-area inorganic oxide having a surface area of about 25 to about 500 m²/g. The porous support preferably is uniform in composition and relatively refractory to the conditions utilized in the process. By the term “uniform in composition,” it is meant that the support be unlayered, has no concentration gradients of the species inherent to its composition, and is completely homogeneous in composition. Thus, if the support is a mixture of two or more refractory materials, the relative amounts of these materials will be constant and uniform throughout the entire support. It is intended to include within the scope of the present invention refractory inorganic oxides such as alumina, titania, zirconia, chromia, zinc oxide, magnesia, thoria, boria, silica-alumina, silica-magnesia, chromia-alumina, alumina-boria, silica-zirconia and other mixtures thereof. The preferred support is substantially free of microcrystalline porous material, i.e., molecular sieves, and in particular contains less than about 1.0 wt.-% of zeolitic materials.

Favored refractory inorganic oxides for use in the present invention comprise one or more of alumina, magnesia, titania, and zirconia, with alumina being particularly favored. Suitable alumina materials are the crystalline aluminas known as the theta-, alpha-, gamma-, and eta-alumina, with theta-, alpha-, and gamma-alumina giving favorable results and theta-alumina being particularly preferred. An especially favored catalyst comprises at least about 80 wt.-% theta alumina. Magnesia, alone or in combination with alumina, comprises an alternative inorganic-oxide component of the catalyst and provides the required nonacidity. The preferred refractory inorganic oxide will have an apparent bulk density of about 0.3 to about 1.1 g/cc and surface area characteristics such that the average pore diameter is about 20 to 1000 angstroms, the pore volume is about 0.05 to about 1 cc/g, and the surface area is about 50 to about 500 m²/g.

It is essential that the catalyst be non-acidic, as acidity lowers the selectivity to para-xylene of the finished catalyst. The required nonacidity may be effected by any suitable method, including impregnation, co-impregnation with a platinum-group metal, or ion exchange. Impregnation of one or more of the alkali and alkaline earth metals, especially potassium, in a salt solution is favored as being an economically attractive method to neutralize the acidity of the support as well as to modify the hydrogenation metal. The alkali or alkaline earth metal is associated with an anion such as hydroxide, nitrate or a halide such as chloride or bromide consistent with nonacidity of the finished catalyst, with a nitrate being favored. Optimally, the support is cold-rolled with an excess of solution in a rotary evaporator in an amount sufficient to provide a nonacidic catalyst. The alkali or alkaline earth metal may be coimpregnated along with a platinum-group metal component, as long as the platinum-group metal does not precipitate in the presence of the salt of the alkali or alkaline earth metal.

Ion exchange is an alternative method of incorporating nonacidity into the catalyst. The inorganic-oxide support is contacted with a solution containing an excess of metal ions over the amount needed to effect nonacidity. Although any suitable method of contacting may be used, an effective method is to circulate a salt solution over the support in a fixed-bed loading tank. A water-soluble metal salt of an alkali or alkaline earth metal is used to provide the required metal ions; a potassium salt is particularly preferred. The support is contacted with the solution suitably at a temperature ranging from about 10° to about 100° C.

The nonacidity of the aromatization-catalyst support may be determined using a variety of methods known in the art. A preferred method of determining acidity is the heptene-cracking test: conversion of heptene, principally by cracking, isomerization and ring formation, is measured at specified conditions, with cracking being particularly indicative of the presence of strong acid sites. Alternatively, nonacidity may be characterized by the ACAC (acetonylacetone) test, in which ACAC is converted over the support to be tested at specified conditions: dimethylfuran in the product is an indicator of acidity, while methylcyclopentenone indicates basicity. Another useful method of measuring acidity is NH₃-TPD (temperature-programmed desorption) as disclosed in U.S. Pat. No. 4,894,142, incorporated herein by reference; the NH₃-TPD acidity strength should be less than about 1.0. Other methods such as ₃₁P solids NMR of adsorbed TMP (trimethylphosphine) also may be used to measure acidity. Suitable methods of characterizing nonacidity are described in more detail in U.S. Pat. No. 5,831,139.

An alternative suitable support having inherent nonacidity may be termed a “synthetic hydrotalcite” characterized as a layered double hydroxide or metal-oxide solid solution. Hydrotalcite is a clay with the ideal unit cell formula of Mg₆Al₂(OH)₁₆(CO₃).4H₂O, and closely related analogs with variable magnesium/aluminum ratios may be readily prepared. These embodiments are solid solutions of a divalent metal oxide and a trivalent metal oxide having the general formula (M⁺²ₓO)(M⁺³_{y}O)OH_{y} derived by calcination of synthetic hydrotalcite-like materials whose general formula may be expressed as (M⁺²)ₓ(M⁺³)_{y}(OH)_{z}A_{q}.rH2O. M^{30 2} is divalent metal or combination of divalent metals selected from the group consisting of magnesium, calcium, barium, nickel, cobalt, iron, copper and zinc. M⁺³ is a trivalent metal or combination of trivalent metals selected from the group consisting of aluminum, gallium, chromium, iron, and lanthanum. Both M⁺² and M⁺³ may be mixtures of metals belonging to the respective class: for example, M⁺² may be pure nickel or may be both nickel and magnesium, or even nickel-magnesium-cobalt; M⁺³ may be solely aluminum or a mixture of aluminum and chromium, or even a mixture of three trivalent metals such as aluminum, chromium, and gallium. A_{q} is an anion, most usually carbonate although other anions may be employed equivalently, especially anions such as nitrate, sulfate, chloride, bromide, hydroxide, and chromate. The ratio x/y of the divalent and trivalent metals can vary between about 2 and about 20, with the ratios of 2 to about 10 being preferred. The case where M⁺² is magnesium, M⁺³ is aluminum, and A is carbonate corresponds to the hydrotalcite series. Calcination of such layered double hydroxides results in destruction of the layered structure and formation of materials which are effectively described as solid solutions of the resulting metal oxides. It is preferable that the (M⁺²ₓO)(M⁺³_{y}O)OH_{y} solid solution has a surface area at least about 150 m²/g, more preferably at least 200 m²/g and it is even more preferable that it be in the range from 300 to 350 m²/g. Preparation of suitable basic metal-oxide supports is described in detail in U.S. Pat. No. 5,254,743.

An inorganic-oxide powder may be formed into a suitable catalyst material according to any of the techniques known to those skilled in the catalyst-carrier-forming art. Spherical carrier particles may be formed, for example, from the preferred alumina by: (1) converting the alumina powder into an alumina sol by reaction with a suitable peptizing acid and water and thereafter dropping a mixture of the resulting sol and a gelling agent into an oil bath to form spherical particles of an alumina gel which are easily converted to a gamma-alumina support by known methods; (2) forming an extrudate from the powder by established methods and thereafter rolling the extrudate particles on a spinning disk until spherical particles are formed which can then be dried and calcined to form the desired particles of spherical support; and (3) wetting the powder with a suitable peptizing agent and thereafter rolling the particles of the powder into spherical masses of the desired size. The powder can also be formed in any other desired shape or type of support known to those skilled in the art such as rods, pills, pellets, tablets, granules, extrudates, and like forms by methods well known to the practitioners of the catalyst material forming art.

The favored form of the preferred non-zeolytic catalyst support is a sphere. Alumina-bound spheres may be continuously manufactured by the well known oil-drop method which comprises: forming an alumina hydrosol by any of the techniques taught in the art and preferably by reacting aluminum metal with hydrochloric acid; combining the resulting hydrosol with the zeolite and a suitable gelling agent; and dropping the resultant mixture into an oil bath maintained at elevated temperatures. The droplets of the mixture remain in the oil bath until they set and form hydrogel spheres. The spheres are then continuously withdrawn from the oil bath and typically subjected to specific aging and drying treatments in oil and an ammoniacal solution to further improve their physical characteristics. The resulting aged and gelled particles are then washed and dried at a relatively low temperature of about 150° to about 205° C. and subjected to a calcination procedure at a temperature of about 450° to about 700° C. for a period of about 1 to about 20 hours. This treatment effects conversion of the alumina hydrogel to the corresponding crystalline gamma-alumina. U.S. Pat. No. 2,620,314 provides basic details and is incorporated herein by reference thereto.

An essential ingredient of the aromatization catalyst is a metal component comprising at least one metal selected from Groups VIII (IUPAC 8-10) and IA (IUPAC 11) of the Periodic Table, including the platinum-group metals, Fe, Co, Ni, Cu, Ag and Au. Of the preferred Group VIII platinum-group metals, i.e., platinum, palladium, rhodium, ruthenium, osmium and iridium, platinum is particularly preferred. Mixtures of platinum-group metals as a uniformly distributed component or platinum-group surface metals also are within the scope of this invention. The platinum-group metal component may exist within the final catalytic composite as a compound such as an oxide, sulfide, halide, or oxyhalide, in chemical combination with one or more of the other ingredients of the composite, or as an elemental metal. Best results are obtained when substantially all of the metals are present in the elemental state. The platinum-group metal component may be present in the final catalyst composite in any amount which is catalytically effective, but relatively small amounts are preferred. The uniformly distributed platinum-group metals generally will comprise from about 0.01 to 5 wt.-% of the final catalyst, and preferably about 0.05 to 2 wt.-%, calculated on an elemental basis.

The preferred platinum-group metal component may be incorporated into the aromatization catalyst in any suitable manner such as coprecipitation or cogellation with the carrier material, ion exchange or impregnation. Impregnation using water-soluble compounds of the metal is preferred. Typical platinum-group compounds which may be employed are chloroplatinic acid, ammonium chloroplatinate, bromoplatinic acid, platinum dichloride, platinum tetrachloride hydrate, tetraamine platinum chloride, tetraamine platinum nitrate, dinitrodiaminoplatinum, platinum dichlorocarbonyl dichloride, palladium chloride, palladium chloride dihydrate, palladium nitrate, and the like. Chloroplatinic acid or tetraamine platinum chloride are preferred as the source of the preferred platinum component.

The aromatization catalyst may contain a halogen component. The halogen component may be either fluorine, chlorine, bromine or iodine or mixtures thereof with chlorine being preferred. Considering the nonacidic nature of the support, the halogen usually is incorporated into the catalyst only in association with the incorporation of a metal component. The halogen component is generally present in a combined state with the inorganic-oxide support. The halogen component is preferably well distributed throughout the catalyst and may comprise from more than 0.2 to about 15 wt.-% calculated on an elemental basis, of the final catalyst.

It is within the scope of the present invention that the aromatization catalyst may contain supplemental metal components known to modify the effect of the preferred platinum component. Such metal modifiers may include one or more of the Group IVB (IUPAC 14) metals, Group 1B (IUPAC 11) metals, rhenium, indium, gallium, bismuth, zinc, uranium, thallium and the rare-earth (lanthanide) metals. Group VIA (IUPAC 6) metals are disfavored, considering the known toxicity of chromium. One or more of tin, indium, germanium, gallium, copper, silver, gold, lead, zinc and the rare-earth elements are favored modifier metals with tin, indium, germanium, cerium and lead being particularly favored. If present, the concentration of a metal modifier in the catalyst may be within the range of 0.001 to 5.0 wt.-%. Catalytically effective amounts of such metal modifiers may be incorporated into the catalyst by any means known in the art.

The final aromatization catalyst generally will be dried at a temperature of from about 100° to 320° C. for about 0.5 to 24 hours, followed by oxidation at a temperature of about 300° to 550° C. in an air atmosphere which preferably contains a chlorine component for 0.5 to 10 hours. Preferably the oxidized catalyst is subjected to a substantially water-free reduction step at a temperature of about 300° to 550° C. for 0.5 to 10 hours or more. The duration of the reduction step should be only as long as necessary to reduce the platinum, in order to avoid pre-deactivation of the catalyst, and may be performed in-situ as part of the plant startup if a dry atmosphere is maintained.

The butene dimer stream contacts the aromatization catalyst in the aromatization zone at aromatization conditions to obtain an aromatized effluent, with the principal reaction being dehydrocyclization of olefinic and paraffinic hydrocarbons to obtain xylenes having a higher-than-equilibrium concentration of para-xylene. Aromatization conditions include a pressure of from about 100 kPa to 6 MPa (absolute), with the preferred range being from 100 kPa to 1 MPa (absolute) and a pressure of about 450 kPa or less at the exit of the last reactor being especially preferred. The volume of the contained aromatization catalyst corresponds to a liquid hourly space velocity of from about 0.5 to 40 hr⁻¹. Free hydrogen as molecular H₂ is supplied to the aromatization zone in an amount sufficient to correspond to a ratio of from about 0.1 to 10 moles of hydrogen per mole of hydrocarbon feedstock; other components of a hydrogen-containing gas stream may comprise one or more of hydrocarbons, nitrogen and steam. The operating temperature, defined as the maximum temperature of the combined hydrocarbon feedstock, free hydrogen, and any components accompanying the free hydrogen, generally is in the range of 260° to 560° C. Hydrocarbon types in the feed stock also influence temperature selection.

In an optional embodiment of the invention, the aromatization zone comprises a hydrogenation reactor to contact the butene dimer with a hydrogenation catalyst to convert olefins to paraffins prior to the aromatization step. The hydrogenation reactor utilizes operating conditions as described above, except that the operating temperature generally is lower and usually is within the range of 150° to 300° C. Suitable hydrogenation catalysts comprise one or more metallic components as an elemental metal or a metal compound. The metals are normally chosen from Groups VIII and IVA of the Periodic Table of the elements such as nickel, platinum, palladium and tin. Platinum is a preferred metal in these catalysts. Based on the weight of the metal, the catalyst may contain from 0.1 to 4.0 wt. % metallic components. The metallic components of the catalyst are supported by a refractory inorganic oxide material such as one of the aluminas, silica, silica-alumina mixtures, various clays and natural or synthetic zeolitic materials. Preferably, the carrier material comprises alumina.

The aromatization process will produce an aromatics-rich effluent stream, with the aromatics content of the C₅+ portion of the effluent typically within the range of about 45 to 95 wt.-%, and more usually more than about 85 wt.-%. The composition of the aromatics will depend principally on the feedstock composition and operating conditions. From the present dimerized isobutene feedstock, the aromatics consist principally of C₈ aromatics with a high para-xylene content.

Using techniques and equipment known in the art, the aromatics-rich effluent from the aromatization zone usually is passed through condensing and cooling facilities to a separator. A hydrogen-rich gas is separated and recycled through suitable compressing means to the first reactor of the aromatization zone, with some net hydrogen available for other uses. The liquid phase from the separation zone is normally withdrawn and processed in a fractionating system.

The aromatization product is fractionated by conventional means to separate C₄ and lighter materials, which may be returned to the light-ends processing section of the dimerization zone in order to recycle butanes to the deisobutanizer. C₅ to C₇ hydrocarbons are removed by fractionation for blending into gasoline or processing in conventional refining units for recovery of benzene and toluene values. Mixed C₈ aromatics, representing a para-xylene concentrate, are recovered overhead in a rerun column, with the bottoms stream, comprising C₉ and heavier aromatics, being a desirable component for blending into premium gasoline. Optionally, the para-xylene concentrate is separated from C₅ to C₇ hydrocarbons and C₉ and heavier aromatics in a sidestream fractionator.

The xylene yield relative to conversion of dimerization product in the aromatization zone generally is at least about 15 wt.-%, and more usually 25 wt.-% or more. A xylene yield of about 35 to 40 wt.-% or more often is attainable in the present process.

In conjunction with the above xylene yields, the concentration of para-xylene in xylenes as represented by the para-xylene concentrate will be significantly above the equilibrium value of 20 to 25 wt.-%. Para-xylene concentration in the xylenes usually will be about 50 wt.-% or more, and often at least about 60 wt.-%. Concentrations of about 70 wt.-% or more of para-xylene in xylenes are achievable, and a concentration of about 85 wt.-% enables ready use of single-stage crystallization for para-xylene recovery.

At least a portion of the para-xylene concentrate is passed to the para-xylene purification zone. This zone comprises any suitable process for recovering high-purity para-xylene product. Suitable processes may include one or more of crystallization, simulated-moving-bed adsorption, pressure-swing adsorption and fractionation. An integrated adsorption and crystallization process is described in U.S. Pat. No. 5,329,060, the provisions of which are incorporated herein by reference. Crystallization, and especially single-stage crystallization, is preferred for para-xylene separation from the para-xylene concentrate of the present invention.

Para-xylene recovery by crystallization from mixed C₈ aromatics is well known. U.S. Pat. Nos. 2,866,833 and 2,985,694 describe multi-stage crystallization processes for para-xylene recovery. Such processes have the disadvantage of low para-xylene recovery due to the formation of eutectic binaries in the mother liquor from which the para-xylene crystals are recovered as well as high operating costs resulting from the multiple stages. U.S. Pat. No. 5,319,060 teaches overcoming this disadvantage by using selective adsorption to enrich the para-xylene feed to crystallization, enabling the use of single-stage crystallization. The relevant contents of the above patents are incorporated herein by reference thereto.

Feed generally enters a crystallizer near the top and exits near the bottom. Each crystallizer is usually equipped with scrapers that remove crystals adhering to the internal walls of the vessel. Crystallizer slurry can be recirculated to the crystallizer to classify the crystals within the crystallizer. Effluent from the crystallizer is passed to a centrifuge, which operates to separate the mother liquor from the para-xylene crystals.

Since the concentration of para-xylene in the mixed C₈ aromatics is relatively high, generally in excess of 70 wt.-% and more usually at least about 85 wt.-%, a purification zone comprising crystallization usually can be reduced to a single stage. This stage can be operated at purification conditions approximating those of the final stage of multi-stage crystallization, for example, temperatures of 0 to −10° C. Chilling usually can be provided by propane vaporization. The crystallization is limited only by the amount of solids that can readily flow in a stream rather than the previously mentioned eutectic limit. At least a portion of the mother liquor can be recycled and mixed with the crystallization feed to provide more liquor to carry additional recovered para-xylene, with the remaining net portion being a desirable component for blending into premium gasoline. Alternatively or in addition, additional para-xylene can be recovered from the mother liquor which may comprise an above-equilibrium concentration of para-xylene.

Optional additional treatment of the stage para-xylene crystals may include washing the crystals with a variety of compounds including but not limited to para-xylene product, normal pentane, toluene, aqueous alcohols and aqueous salts to improve final product purity by removing adhering second stage mother liquor. After melting the crystals, it may be necessary to feed the resulting mixture to a fractionation column to separate the para-xylene product from the wash liquor.

The high-purity para-xylene recovered from the purification zone comprises at least about 99.5 wt.-% para-xylene, and preferably at least about 99.7 wt.-% para-xylene.

Other embodiments and variants encompassed by and within the spirit of the present invention as claimed will be apparent to the skilled routineer. Examples follow which illustrate certain specific embodiments, and these particularly should not be construed to limit the scope of the invention as set forth in the claims.

### EXAMPLES

### Example 1

A catalyst comprising 18.6 wt.-% Cr and 3.41 wt.-% K on a gamma-alumina support was prepared following the procedure described in DuPont's patent application US0015026 A1. This catalyst serves as a comparative example of the known art for converting 2,2,4-trimethylpentane or 2,4,4-trimethylpentene to para-xylene, and is designated Catalyst X.

### Example 2

A gamma-alumina sphere comprising 0.3 wt.-% Sn was impregnated with chloroplatinic acid (CPA) and 2 wt.-% HCl to give 0.29 wt.-% Pt. The impregnated support then was dried, oxychlorinated in the presence of air and HCl, and finally reduced in the presence of H₂. This catalyst of the known art is designated Catalyst Y.

### Example 3

A theta-alumina sphere of 1/16-inch diameter prepared as described hereinabove and comprising 0.2 wt.-% Sn was impregnated with KCl and chloroplatinic acid (CPA) to give 0.45 wt.-% Pt and 0.70 wt.-% K. The impregnated support then was air calcined, conditioned in the presence of HCl and Cl₂, and finally reduced in the presence of H₂. This catalyst is designated as Catalyst A.

### Example 4

A theta -alumina sphere of 1/16-inch diameter and comprising 0.2 wt.-% Sn was impregnated with KCl and chloroplatinic acid (CPA) to give 0.45 wt.-% Pt and 1.50 wt.-% K, and finished following the procedure described in Example 3. This catalyst is designated as Catalyst B.

### Example 5

A theta -alumina sphere of 1/16-inch diameter and comprising 0.2 wt.-% Sn was impregnated with CsNO₃ and tetraamineplatinum nitrate (TAPN) to give 0.45 wt.-% Pt and 2.38 wt.-% Cs, and finished following the procedure described in Example 3. This catalyst is designated as Catalyst C.

### Example 6

A gamma-alumina sphere of 1/32-inch diameter and comprising 0.57 wt.-% Sn was impregnated with KNO₃ and tetraamineplatinum nitrate (TAPN) to give 0.72 wt.-% Pt and 1.30 wt.-% K, and finished following the procedure described in Example 3. This catalyst is designated as Catalyst D.

### Example 7

A theta-alumina sphere of approximately 1/32-inch diameter and comprising 0.57 wt.-% Sn was impregnated with KNO₃ and tetraamineplatinum nitrate (TAPN) to give 1.26 wt.-% and 0.70 wt.-% K, and finished following the procedure described in Example 3. This catalyst is designated as Catalyst E.

### Example 8

Catalysts X and Y of the known art (“art”) and catalysts A, B, C, D and E of the present invention (“inv.”) were sized into 20×40 mesh and were tested in the micro-reactor at atmospheric pressure with results as shown in Table 1. The catalysts, in reactor loadings from 250 to 1000 mg, were pre-reduced in the presence of H₂ at 450° C. The reactor was cooled to 300° C. and H₂ flow was directed through a bath of 2,2,4-trimethylpentane (“TMP”) or 2,4,4-trimethylpentene (“TMP=”) as indicated. The catalyst performance then was recorded at 500 and 550° C. based on data from non-polar and polar GC columns. The results show the catalysts of the present invention were significantly more effective than the catalysts of the known art in providing a combination of high xylene yields and high para-xylene content of the xylene product. None of the catalysts of the known art achieved a combination of 20% or higher xylene yield relative to conversion and 65% or higher para-xylene in the xylene product, levels which were achieved by all of the catalysts of the invention.

TABLE 1

| Catalyst | | | Temp. | Conversion | Xylenes | B/T/EB | C₁-C₇ | C₄+ = C₄ | Xylenes/ | P-xylene/ |
|---|---|---|---|---|---|---|---|---|---|---|
| Desig. | mg | Feed* | ° C. | wt.-% | wt.-% | wt.-%^{#} | wt.-%\hat{ } | wt.-%^{&} | Conversion | Xylenes |
| X (art) | 500 | TMP | 500 | 7.48 | 0.14 | 0.46 | 6.74 | 5.27 | 1.9% | 79.7% |
| X (art) | 500 | TMP | 550 | 25.63 | 2.64 | 3.96 | 20.99 | 15.44 | 10.3% | 90.4% |
| A (inv.) | 250 | TMP | 500 | 18.31 | 3.99 | 4.50 | 8.93 | 4.98 | 21.8% | 74.0% |
| A (inv.) | 250 | TMP | 550 | 31.12 | 8.88 | 10.40 | 15.27 | 10.76 | 28.5% | 65.9% |
| A (inv.) | 1000 | TMP | 500 | 34.18 | 8.29 | 9.13 | 17.92 | 11.72 | 24.3% | 74.0% |
| A (inv.) | 1000 | TMP | 550 | 48.94 | 18.24 | 20.41 | 22.84 | 17.57 | 37.3% | 68.8% |
| X (art) | 1000 | TMP= | 500 | 34.17 | 0.73 | 1.64 | 30.63 | 24.89 | 2.1% | 62.5% |
| X (art) | 1000 | TMP= | 550 | 62.02 | 7.38 | 9.82 | 50.92 | 40.56 | 11.9% | 84.0% |
| Y (art) | 1000 | TMP= | 500 | 99.80 | 29.42 | 39.83 | 49.06 | 39.08 | 29.5% | 23.6% |
| Y (art) | 1000 | TMP= | 550 | 99.94 | 28.87 | 43.14 | 32.98 | 17.99 | 28.9% | 22.1% |
| A (inv.) | 1000 | TMP= | 500 | 32.19 | 13.91 | 14.63 | 11.90 | 4.01 | 43.2% | 70.9% |
| A (inv.) | 1000 | TMP= | 550 | 42.26 | 17.41 | 18.63 | 18.06 | 13.27 | 41.2% | 73.4% |
| B (inv.) | 1000 | TMP= | 550 | 37.09 | 9.20 | 9.93 | 21.90 | 18.38 | 24.8% | 77.6% |
| C (inv.) | 1000 | TMP= | 500 | 27.19 | 9.69 | 10.35 | 11.68 | 3.51 | 35.6% | 68.1% |
| C (inv.) | 1000 | TMP= | 550 | 42.74 | 15.88 | 17.17 | 19.17 | 14.24 | 37.2% | 72.3% |
| D (inv.) | 1000 | TMP= | 500 | 37.22 | 13.42 | 14.33 | 15.63 | 4.95 | 36.1% | 67.7% |
| D (inv.) | 1000 | TMP= | 550 | 52.23 | 23.80 | 25.49 | 19.77 | 14.14 | 45.6% | 69.2% |
| E (inv.) | 1000 | TMP= | 500 | 43.19 | 21.58 | 22.72 | 15.45 | 6.72 | 50.0% | 68.6% |
| E (inv.) | 1000 | TMP= | 550 | 52.24 | 24.75 | 26.55 | 20.06 | 14.57 | 47.4% | 70.2% |

*TMP is 2,2,4-trimethylpentane, TMP= is 2,4,4-trimethylpentene
^{#}BTX is benzene, toluene and ethylbenzene
\hat{ }C₁ to C₇ paraffins and olefins
^{&}butanes + butenes, included in C₁-C₇

### Example 9

A catalyst of the art as found in the literature was prepared for comparison with selected catalysts in pilot-plant tests. Theta-alumina containing 0.3 wt % tin was impregnated to give 3.5 wt % Cr and 1.3 wt % of K. The catalyst is designated as Catalyst W.

### Example 10

Catalyst A of the invention as previously described was tested further in comparison to Catalyst W in a laboratory pilot plant under different sets of process conditions. In this test 10 ml of catalyst was loaded into a stainless-steel reactor. The catalyst was pre-reduced in H₂ flow at 450° C. for 2 hours. The reactor then was cooled to 300° C. and 2,2,4-trimethylpentane was introduced. Typical operating conditions comprised a plant pressure of 155 to 315 kPa, H₂ to hydrocarbon molar ratio of 1 to 4 and temperatures of about 500° to 560° C. as indicated. Products were analyzed by polar and non-polar GC columns to obtain the component breakdown. Results as shown in Table 2 demonstrated that Pt—Sn—K supported on alumina (Catalyst A) is active and selective in converting 2,2,4-trimethylpentane to xylene with minimal formation of byproducts in comparison with the catalyst of the known art, and that the resulting xylenes have a para-xylene concentration significantly higher than that calculated based on thermodynamics.

TABLE 2

| Catalyst | Press. | Temp. | Conversion | Xylenes | B/T/EB | C₁-C₇ | C₄+ = C₄ | Xylenes/ | P-xylene/ |
|---|---|---|---|---|---|---|---|---|---|
| Desig. | Kpa | ° C. | wt.-% | wt.-% | wt.-%^{#} | wt.-%* | wt.-%^{&} | Conversion | Xylenes |
| W (art) | 155 | 552 | 36.7 | 4.9 | 5.1 | 30.6 | 28.6 | 13.4% | 87.8% |
| A (inv.) | 315 | 559 | 88.6 | 31.2 | 34.8 | 39.0 | 31.5 | 35.2% | 60.8% |
| A (inv.) | 315 | 541 | 71.8 | 20.8 | 22.3 | 29.9 | 23.6 | 29.0% | 80.1% |
| A (inv.) | 200 | 512 | 43.9 | 21.0 | 28.6 | 6.3 | 5.2 | 47.8% | 75.2% |

^{#}BTX is benzene, toluene and ethylbenzene
*C₁ to C₇ paraffins and olefins
^{&}butanes + butenes, included in C₁-C₇

1. A process for the production of high-purity para-xylene from a butene dimer by contacting the butene dimer with a non-zeolitic and nonacidic aromatization catalyst in an aromatization zone operating at aromatization conditions to produce a para-xylene concentrate comprising xylenes having a higher-than-equilibrium content of para-xylene.

2. The process of claim 1 wherein the aromatization conditions comprise a pressure of from about 100 kPa to 6 MPa (absolute), a hydrogen to hydrocarbon ratio of from about 0.1 to 10, a liquid hourly space velocity of from about 0.5 to 40 hr⁻¹, and an operating temperature of from about 260° to 560° C.

3. The process of claim 1 wherein the aromatization catalyst comprises:
(a) a support comprising an oxide of a metal selected from one or more of alumina, titania and zirconia;
(b) a hydrogenation metal selected from one or more of the platinum-group metals;
(c) a metal modifier selected from one or more of tin, indium, germanium, gallium, copper, silver, gold, lead, zinc and the rare-earth elements; and,
(d) one or more of the alkali and alkaline earth metals.

4. The process of claim 1 wherein the aromatization catalyst comprises the substantial absence of a Group VIB (6) metal.

5. The process of claim 3 wherein the support comprises at least about 80 wt.-% theta alumina.

6. The process of claim 1 wherein the xylene yield relative to conversion of butene dimer is at least about 15 wt.-% and the concentrate of para-xylene in the para-xylene concentrate is at least about 50 wt.-%.

7. The process of claim 1 wherein the xylene yield relative to conversion of butene dimer is at least about 15 wt.-% and the concentrate of para-xylene in the para-xylene concentrate is at least about 60 wt.-%

8. A process combination for the production of high-purity para-xylene from a butene dimer comprising:
(a) contacting at least a portion of the butene dimer with an aromatization catalyst in an aromatization zone operating at aromatization conditions produce an para-xylene concentrate comprising xylenes having a higher-than-equilibrium content of para-xylene; and,
(b) passing at least a portion of the para-xylene concentrate to a para-xylene purification zone operating at purification-zone conditions to recover high-purity para-xylene.

9. The process combination of claim 8 wherein the aromatization catalyst comprises:
(a) a support comprising an oxide of a metal selected from one or more of alumina, titania and zirconia;
(b) a hydrogenation metal selected from one or more of the platinum-group metals;
(c) a metal modifier selected from one or more of tin, indium, germanium, gallium, copper, silver, gold, lead; zinc and the rare-earth elements; and,
(d) one or more of the alkali and alkaline earth metals.

10. The process combination of claim 8 wherein the aromatization catalyst comprises the substantial absence of a Group VIB (6) metal.

11. The process combination of claim 9 wherein the support comprises at least about 80 wt.-% theta alumina.

12. The process combination of claim 8 wherein the xylene yield relative to conversion of butene dimer is at least about 15 wt.-% and the concentrate of para-xylene in the para-xylene concentrate is at least about 50 wt.-%.

13. The process combination of claim 8 wherein the high-purity paraxylene comprises at least about 99.7 wt.-% para-xylene.

14. A process combination for the production of high-purity para-xylene from an isobutene-rich feed comprising:
a) contacting the isobutene-rich feed with a dimerization catalyst in a dimerization zone operating at dimerization conditions to produce a butene dimer comprising one or both of C₈ isoolefins and C₈ isoparaffins;
b) contacting at least a portion of the butene dimer with an aromatization catalyst in an aromatization zone operating at aromatization conditions produce an para-xylene concentrate comprising xylenes having a higher-than-equilibrium content of para-xylene; and,
c) passing at least a portion of the para-xylene concentrate to a para-xylene purification zone operating at purification-zone conditions to recover high-purity para-xylene.

15. The process combination of claim 14 wherein the dimerization catalyst of step (a) comprises a cationic resin.

16. The process combination of claim 14 wherein the dimerization catalyst of step (a) comprises solid phosphoric acid.

17. The process combination of claim 14 wherein step (a) comprises contacting the dehydrogenation effluent stream and an isobutane-containing stream with the dimerization catalyst which comprises an alkylation catalyst in the dimerization zone which comprises alkylation to produce a butene dimer which comprises a high concentration of C₈ isoparaffins.

18. The process combination of claim 14 wherein the aromatization catalyst comprises:
(a) a support comprising an oxide of a metal selected from one or more of alumina, titania and zirconia;
(b) a hydrogenation metal selected from one or more of the platinum-group metals;
(c) a metal modifier selected from one or more of tin, indium, germanium, gallium, copper, silver, gold, lead, zinc and the rare-earth elements; and,
(f) one or more of the alkali and alkaline earth metals.

19. The process combination of claim 14 wherein the aromatization catalyst comprises the substantial absence of a Group VIB (6) metal.

20. The process combination of claim 18 wherein the support comprises at least about 80 wt.-% theta alumina.
